(19) United States
(12) Patent Application Publication
(10) Pub. No.: US 2007/0166246 A1
(43) Pub. Date: Jul. 19, 2007

(54) Composition for oral cavity

(75) Inventors: Kinya Takagaki, Fukuoka (JP); Sadao Mori, Fukuoka (JP)

Correspondence Address: AMIN, TUROCY & CALVIN, LLP, 1900 EAST 9TH STREET, NATIONAL CITY CENTER, 24TH FLOOR,, CLEVELAND, OH, 44114, US

(73) Assignee: TOYO SHINYAKU CO., LTD., Fukuoka-shi, Fukuoka (JP)

(21) Appl. No.: 10/599,014

(22) PCT Filed: Mar. 2, 2005
(86) PCT No.: PCT/JP05/04035

§ 371 Date: Sep. 18, 2006

(30) Foreign Application Priority Data
Mar. 19, 2004 (JP) 2004-081710

(51) Int. Cl. A61K 8/97; A61K 36/13

(52) U.S. Cl. 424/58; 424/770

(57) ABSTRACT

The present invention provides a composition for the oral cavity that comprises a pine bark extract that is extracted from the bark of a pine. The composition for the oral cavity of the present invention can provide a plurality of effects such as an effect of improving blood flow in the mouth and an effect of reducing halitosis.

### TECHNICAL FIELD

The present invention relates to a composition for the oral cavity that contains a pine bark extract that is extracted from the bark of a pine, and in particular, relates to a composition for the oral cavity having effects such as an effect of improving blood flow in the mouth.

### BACKGROUND ART

Recently, plant extracts that contain proanthocyanidins have attracted attention for their antioxidation properties and the like. Among such plant extracts, an extract that is extracted from the bark of a pine (pine bark extract) is brought to particular attention because it is known to have, in addition to the antioxidation properties, tyrosinase inhibitory properties, collagenase inhibitory properties, DNA protection properties, and ability of improving the condition of being overly sensitive to the cold (see Japanese Laid-Open Patent Publication Nos. 2003-238425, 2003-238426, 2003-238427, and 2003-238428).

### DISCLOSURE OF INVENTION

It has been recently expected that various functions are added to products that are used in the human oral cavity (compositions for the oral cavity), such as candies, tablets, troches, and mouthwashes. Examples of such functions include a function for preventing periodontal diseases and a function of reducing halitosis.

However, there is a problem in that when it is intended to obtain a plurality of effects such as the function for preventing periodontal diseases (such as an effect of improving blood flow in the mouth) and an effect of reducing halitosis with a composition for the oral cavity, it is necessary to add substances each of which can provide one of these effects separately. For example, when it is intended to obtain the effect of improving blood flow in the mouth and the effect of reducing halitosis with a mouthwash, it is necessary to add to a solvent a substance providing the effect of improving blood flow in the mouth and a substance providing the effect of reducing halitosis separately.

The present invention was arrived at in view of this problem, and it is an object thereof to provide a composition for the oral cavity that can provide a plurality of effects such as the effect of improving blood flow in the mouth and the effect of reducing halitosis, by adding one substance to a composition for the oral cavity.

In view of this problem, the inventors of the present invention conducted in-depth research on various substances. As a result, it was found that an extract that is extracted from the bark of a pine (pine bark extract) has a plurality of effects required for a composition for the oral cavity, such as the effect of improving blood flow in the mouth and the effect of reducing halitosis, and thus the present invention was achieved.

The composition for the oral cavity of the present invention comprises a pine bark extract that is extracted from the bark of a pine.

The composition for the oral cavity of the present invention is prepared such that at least 2 mg of the pine bark extract is administered to the oral cavity at one time. For example, it is preferable that the composition is prepared such that at least 2 mg of the pine bark extract is administered to the human oral cavity at one time. Furthermore, it is preferable that at least 2 mg of the pine bark extract is contained in an amount of the composition that is suitable for administration to the human oral cavity at one time. For example, when the composition for the oral cavity is a solid, it may be a solid composition that can be dissolved in the human oral cavity and contains at least 2 mg of the pine bark extract in an amount of the solid that is suitable for administration to the human oral cavity.

In addition to the above, the composition for the oral cavity of the present invention is prepared such that at least 2 mg of the pine bark extract is administered to the oral cavity at one time.

In addition to the above, the composition for the oral cavity of the present invention further comprises a solvent and the concentration of the pine bark extract is at least 0.2 (g/L).

The composition for the oral cavity of the present invention contains a pine bark extract that is extracted from the bark of a pine. This composition for the oral cavity can provide a plurality of effects required for a composition for the oral cavity, such as the effect of improving blood flow in the mouth and the effect of reducing halitosis, by adding one substance, namely the pine bark extract. In particular, when the pine bark extract contains at least 20 wt % of oligomeric proanthocyanidins with respect to the dry weight of the pine bark extract, the effects are increased even more.

### BEST MODE FOR CARRYING OUT THE INVENTION

Hereinafter, the composition for the oral cavity of the present invention is described. It should be noted that the present invention is not to be interpreted as being limited to the following descriptions and various changes are possible within the scope of the invention.

(Pine Bark Extract)

The composition for the oral cavity of the present invention contains an extract that is extracted from the bark of a pine (pine bark extract). First, this pine bark extract will be described. A pine bark extract refers to an extract that can be obtained using the bark of a pine. There is no specific limitation regarding a method for obtaining a pine bark extract. For example, a pine bark extract may be obtained by methods as described in Japanese Laid-Open Patent Publication Nos. 2003-238425, 2003-238426, 2003-238427, and 2003-238428. In the present invention, it is preferable to use a pine bark extract that abundantly contains proanthocyanidins.

Examples of pines for obtaining pine bark that can be employed in order to obtain the pine bark extract include French maritime pine (Pinus martima), Larix leptolepis, Pinus thunbergii, Pinus densiflora, Pinus parviflora, Pinus pentaphylla, Pinus koraiensis, Pinus pumila, Pinus luchuensis, utsukushimatsu (Pinus densiflora form. umbraculifera), Pinus palustris, Pinus bungeana, and Anneda in Quebec, Canada. Among such plurality of pines, French maritime pine (Pinus martima) is preferably used in the present invention. That is, the pine bark extract is preferably obtained from the bark of French maritime pine. When the bark of French maritime pine is selected as the bark used for the extraction, the pine bark extract that contains proanthocyanidins abundantly can be obtained stably and definitely. Moreover, when the bark of French maritime pine is selected, the pine bark extract that contains oligomeric proanthocyanidins (hereinafter, referred to ‘OPCs’ suitably) in high concentration (e.g., at least 20 wt % of OPCs with respect to the dry weight of the pine bark extract) can be produced stably and definitely. Examples of the methods for obtaining the pine bark extract from the bark of French maritime pine include methods described in Japanese Laid-Open Patent Publication Nos. 2003-238425, 2003-238426, 2003-238427, and 2003-238428.

French maritime pine (Pinus martima) refers to maritime pines that grow in a part of the Atlantic coastal area in southern France. The bark of this French maritime pine contains proanthocyanidins (in particular, OPCs), organic acids, and other bioactive substances, and proanthocyanidins, which are the main component of the French maritime pine bark, are known to have a potent antioxidation ability of removing active oxygen.

In this specification, among proanthocyanidins, condensation products having flavan-3-ol and/or flavan-3,4-diol as a constituent unit and having a degree of polymerization of 2 to 4 are referred to as oligomeric proanthocyanidins (OPCs). OPCs, which are one type of polyphenol, are potent antioxidants produced by plants, and are contained concentratedly in portions of plant leaves, bark, or skin or seeds of fruits. More specifically, they are contained in the bark of pine; the fruit or seeds of grape, blueberry, strawberry, avocado, locust, and cowberry; barley, wheat, soybean, black soybean, and cacao; the inner skin of peanuts; and the leaves of ginkgo, for example. Moreover, it is known that OPCs are also contained in cola nuts in West Africa, the roots of Rathania in Peru, and Japanese green tea. OPCs cannot be produced in the human body.

In this specification, proanthocyanidins are group of compounds that give anthocyanidins when proanthocyanidins are subjected to chemical treatments (e.g., acid treatment). More specifically, proanthocyanidins are group of compounds that are condensation products having flavan-3-ol and/or flavan-3,4-diol as a constituent unit and having a degree of polymerization of 2 or more.

The pine bark extract contained in the composition for the oral cavity of the present invention preferably contains at least 20 wt % of oligomeric proanthocyanidins (OPCs) with respect to the dry weight of the pine bark extract. Thus, by using the pine bark extract that contains a large amount of OPCs, the composition for the oral cavity of the present invention has a plurality of effects described in the examples below. That is, the pine bark extract that contains at least 20 wt %, preferably at least 25 wt %, more preferably at least 30 wt %, even more preferably at least 40 wt %, and most preferably at least 50 wt % of OPCs with respect to the dry weight of the pine bark extract are used for the composition for the oral cavity of the present invention.

(Methods for Producing the Pine Bark Extract)

Hereinafter, methods for producing the extract (pine bark extract) used for the composition for the oral cavity will be described. The pine bark extract that can be used for the present invention can be specifically prepared by the following method. However, the following method is merely an example, and the pine bark extract used for the present invention is not limited to the extract obtained by the following method.

The pine bark extract is obtained by extracting the bark of the above-mentioned pines using water or an organic solvent. When water is used, cold water, warm water, or hot water are employed. In order to increase the extraction efficiency, a salt such as sodium chloride may be added to the water.

As the organic solvent that can be employed for extraction, an organic solvent that is acceptable for production of foods or pharmaceuticals can be employed. Examples of such solvent include methanol, ethanol, 1-propanol, 2-propanol, 1-butanol, 2-butanol, acetone, hexane, cyclohexane, propylene glycol, aqueous ethanol, aqueous propylene glycol, methyl ethyl ketone, glycerin, methyl acetate, ethyl acetate, diethyl ether, dichloromethane, edible oils or fats, 1,1,1,2-tetrafluoroethane, and 1,1,2-trichloroethene. The water and the organic solvents may be used alone or in combination. In particular, water or polar solvent are preferable. Among them, hot water, aqueous ethanol, and aqueous propylene glycol are preferably used.

The method for obtaining the extract from pine bark is not particularly limited, and heat extraction or supercritical fluid extraction can be employed, for example.

Supercritical fluid extraction is a method for performing extraction using a supercritical fluid. A supercritical fluid is in a state that is above the liquid-vapor critical point in the phase diagram showing critical temperature and critical pressure. Examples of compounds that can be employed as a supercritical fluid include carbon dioxide, ethylene, propane, and nitrous oxide (laughter gas). Carbon dioxide is preferably used.

Supercritical fluid extraction includes an extraction step in which a target component is extracted with a supercritical fluid and a separation step in which the target component is separated from the supercritical fluid. In the separation step, any separation process can be employed, examples of which include a separation based on a change in pressure, a separation based on a change in temperature, and a separation using an adsorbent or absorbent.

Moreover, it is also possible to perform supercritical fluid extraction in which an entrainer is added. In this method, extraction is performed using an extracting fluid obtained by adding, for example, ethanol, propanol, n-hexane, acetone, toluene, or another aliphatic lower alcohol, aliphatic hydrocarbon, aromatic hydrocarbon, or ketone at about 2 to 20 W/V % to a supercritical fluid, so that the solubility of a target substance to be extracted, such as OPCs and catechins (described later), in the extracting fluid is dramatically increased or the selectivity of separation is enhanced. Thus, a pine bark extract is obtained efficiently.

Since supercritical fluid extraction can be performed at a relatively low temperature, it has the following advantages: it is applicable for extracting substances that deteriorate or decompose at high temperatures; the extracting fluid does not remain; and the extracting fluid can be recovered and recycled, so that a step of removing the extracting fluid and the like can be omitted, and thus, the process can be simplified.

Furthermore, methods other than those mentioned above can be employed for extraction from pine bark, and the examples of which include a batch method using liquid carbon dioxide, a reflux method using liquid carbon dioxide, a reflux method using supercritical carbon dioxide, and the like. The pine bark extract may be obtained by employing a combination of a plurality of extraction processes to perform extraction from pine bark. By combining a plurality of extraction processes, pine bark extracts with various components can be obtained.

(Composition for the Oral Cavity)

Next, the composition for the oral cavity will be described. In the present invention, a composition for the oral cavity generally refers to a product that is used in the oral cavity and exerts a certain effect in the oral cavity. Examples of the certain effect exerted in the oral cavity herein include the effect of reducing halitosis, the effect of preventing periodontal diseases, and the effect of improving (raising) blood flow in the mouth.

Furthermore, it is preferable that the composition for the oral cavity of the present invention has a plurality of effects (certain effects exerted in the oral cavity), when the composition (in particular, an active component thereof (i.e., pine bark extract)) is applied to cells in the mouth. For example, if the composition for the oral cavity of the present invention has the effect of reducing halitosis and the effect of improving (raising) blood flow in the mouth, then it can be used as an agent having two effects, namely the effect of reducing halitosis and the effect of improving blood flow in the mouth.

As described above, the composition for the oral cavity is used in the oral cavity. In one example of use, the composition for the oral cavity is allowed to be present in the oral cavity for a predetermined time. Regarding the form in which the composition for the oral cavity is allowed to be present in the oral cavity, the following cases can be exemplified: when the composition for the oral cavity is a liquid, a form in which the mouth is rinsed with the composition for the oral cavity, a form in which the composition for the oral cavity is put in the mouth for a predetermined time and then ejected, or the like can be employed. When the composition for the oral cavity is a solid (such as tablets, powders, and granules), as the form in which the composition for the oral cavity is allowed to be present in the oral cavity, for example, a form in which the composition for the oral cavity is dissolved in the oral cavity can be employed. When the composition for the oral cavity is a solid, the amount of the pine bark extract that is released may be controlled by absorbing the pine bark extract to a carrier or to a carrier crosslinked by using a crosslinking agent, in order to adjust the degree of the effect or the duration of the effect.

Specific examples of the form of the composition for the oral cavity include dentifrices (such as liquid dentifrices, toothpastes, and tooth powders), mouthwashes, gargles, mouth refreshers, and tablets. It would be appreciated that the composition for the oral cavity may be in the form of a food product such as troches, candies, gum, and gummies. Furthermore, it is preferable that the composition for the oral cavity of the present invention has a plurality of effects when the composition for the oral cavity (in particular, the pine bark extract, which is an active component) is applied to cells in the mouth. Thus, a form in which the composition for the oral cavity (in particular, an active component thereof) can be applied to cells in the oral cavity for a predetermined time is preferable. It should be noted that the lower limit value of the “predetermined time” herein is one second or more, preferably 30 seconds or more, more preferably one minute or more, and even more preferably five minutes or more. The upper limit value of the “predetermined time” is 30 minutes or less, preferably 15 minutes or less, and more preferably 10 minutes or less. Thus, as the form of the composition for the oral cavity, for example, a form in which the composition can be chewed or licked in the oral cavity (specifically, tablets, troches, gum, and the like) or a form in which the composition can be easily put in the oral cavity (specifically, mouthwashes, dentifrices, and the like) is preferable. More specifically, a form in which the pine bark extract can be applied to cells in the mouth for a comparatively long time and in which halitosis can be reduced when food, such as garlic, that has a strong odor is ingested is more preferable. Examples of this form include a form in which the composition for the oral cavity is gradually dissolved in the oral cavity and a form in which the composition for the oral cavity is chewed in the mouth, and the form of tablets, troches, or the like is more preferable.

Furthermore, the “effect of improving (raising) blood flow in the mouth” described above refers to an effect of raising blood flow in a tissue (such as the gingiva) of the mouth. More specifically, it refers to an effect that when the composition for the oral cavity of the present invention is allowed to be present in the oral cavity, blood flow in a tissue of the mouth is raised to be higher than that before the composition is allowed to be present in the oral cavity. Furthermore, the “effect of reducing halitosis” described above refers to an effect that when the composition for the oral cavity of the present invention is allowed to be present in the oral cavity, an odor emitted from the mouth is reduced to be smaller than that before the composition is allowed to be present. Examples of the “reducing an odor emitted from the mouth” herein preferably include reducing an odor that is unpleasant for people, such as garlic odor.

As described above, the composition of the present invention may contain various components, if necessary. The content of the various components may be freely determined. Examples of the various components include components (such as polishing agents, thickening agents, binding agents, foaming agents and foaming aids, antiseptics and disinfectants, sweetening agents, solvents, coloring agents (pigments), flavors, and various active components) that can be contained in ordinary compositions for the oral cavity, or components (such as base materials, and extracts from animals or plants) that can be usually contained in quasi-drugs, cosmetics, and toiletries. These components may be contained alone or in combination.

Specific examples of the various components include plant antibacterial extracts such as oil soluble licorice essences and mulberry bark essences; vitamins such as vitamin A, vitamin C, vitamin E, and derivatives thereof; sugar alcohols having four or five carbon atoms such as erythritol, maltitol, and xylitol; and catechins such as tea extracts.

Among these, vitamin E, xylitol, erythritol, and tea extracts are preferable. By the use of a sugar alcohol, it is possible to obtain a composition for the oral cavity that not only provides a refreshed feeling in the mouth but also reduces the astringent taste of the pine bark extract and is suitable also in terms of the palatability.

Furthermore, examples of the polishing agents include silica-based polishing agents (e.g., precipitated silica, silica gel, aluminosilicate, and zirconosilicate), aluminium hydroxide, calcium hydrogenphosphate dihydrate and an anhydride thereof, calcium pyrophosphate, sodium metaphosphate, hydroxyapatite, heavy and light calcium carbonate, zirconium silicate, alumina, magnesium carbonate, and synthetic resin-based polishing agents. Examples of the thickening agents and the binding agents include glycerin, sorbitol, propylene glycol, polyethylene glycol, maltitol, carboxymethylcellulose sodium, carrageenan, sodium alginate, sodium polyacrylate, carbopole, hydroxyethyl cellulose, hydroxypropyl cellulose, methyl cellulose, montmorillonite, guar gum, veegum, karaya gum, gum arabic, locust bean gum, gelatin, polyvinyl alcohol, polyvinyl pyrrolidone, copolymers of methoxyethylene and maleic anhydride, laponite, and viscosity-increasing silica.

Examples of the foaming agents and the foaming aids include anionic surfactants such as fatty acid surfactants, linear alkylbenzene surfactants, alpha olefin surfactants, normal paraffin surfactants, or higher alcohol surfactants; nonionic surfactants such as sucrose fatty acid ester, fatty acid alkanolamide, polyglycerin fatty acid ester, polyoxyethylene alkyl ether, polyoxyethylene fatty acid ester, polyoxyethylene hydrogenated castor oil, polyoxyethylene polyhydric alcohol fatty acid ester, sorbitan fatty acid ester, and polyoxyethylene polyoxypropylene copolymer; amphoteric surfactants such as imidazoline surfactants and betaine surfactants; cationic surfactants such as and amine surfactants and quaternary ammonium salt surfactants.

Examples of the antiseptics and the disinfectants include esters of para-hydroxybenzoic acid, benzoic acid and salts thereof, salicylic acid and salts thereof, p-methoxycinnamaldehyde, chlorhexidine hydrochloride, chlorhexidine gluconate, lower fatty acid monoglycerides, cetylpyridinium chloride, benzethonium chloride, benzalkonium chloride, isopropyl methylphenol, and triclosan.

Furthermore, sweetening agents such as saccharin sodium, stevia extract, stevioside, neohesperidine dihydrochalcone, perillartine, aspartyl phenylalanine methylester, thaumatin, paratinose, and licorice powder, solvents such as water, ethanol, isopropyl alcohol, and cetanol, and coloring agents can be used.

Examples of the flavors include natural flavors such as peppermint oil, spearmint oil, anise oil, eucalyptus oil, wintergreen oil, cassia oil, clove oil, thyme oil, sage oil, lemon oil, orange oil, peppermint oil, cardamom oil, coriander oil, mandarine oil, lime oil, lavender oil, rosemary oil, laurus oil, chamomile oil, caraway oil, marjoram oil, bay oil, lemonglass oil, origanum oil, and pineneedle oil; flavors such as menthol, carvone, anethole, cineole, methyl salicylate, cinnamaldehyde, eugenol, thymol, linalool, linalyl acetate, limonene, menthone, menthyl acetate, pinene, octyl aldehyde, citral, pulegone, carvyl acetate, and anisaldehyde; flavors and/or natural flavors such as ethyl acetate, ethyl butyrate, allyl cyclohexanepropionate, methylanthranilate, ethyl methylphenylglycidate, vanillin, undecalactone, hexanal, ethyl alcohol, propyl alcohol, butanol, and isoamyl alcohol; and flavors containing the above described flavors, such as strawberry flavor, apple flavor, banana flavor, pineapple flavor, grape flavor, mango flavor, tropical fruit flavor, butter flavor, milk flavor, and fruit-mix flavor. It is also possible to use known flavors that are used in compositions for the oral cavity.

In addition to the above, active components can be contained. Examples of the active components include fluorides such as sodium fluoride, stannous fluoride, potassium fluoride, ammonium fluoride, and sodium monofluorophosphate; and anti-plasmin agents such as tranexamic acid and ε-aminocaproic acid.

Furthermore, as components used in the form (specifically, tablets, troches, gum, and the like) that can be chewed or licked in the mouth, α-cyclodextrin, β-cyclodextrin, liquid paraffin, microcrystalline wax, paraffin wax, starch, corn starch, lactose, powdered sugar, gum base, and starch syrup, for example, also can be contained, in addition to the components above.

Next, the content of the pine bark extract in the composition for the oral cavity will be described. The composition for the oral cavity of the present invention is prepared such that the amount of the pine bark extract that is administered to the oral cavity (preferably, to the human oral cavity) at one time is at least 2 mg. It would be appreciated that the composition for the oral cavity may be prepared such that the amount of the pine bark extract is at least 3 mg, and more preferably at least 4 mg. Furthermore, the composition for the oral cavity may be prepared such that the amount of the composition for the oral cavity is suitable for administration to the human oral cavity at one time, wherein the lower limit value of the content of the pine bark extract is at least 2 mg, preferably at least 3 mg, and more preferably at least 4 mg.

The upper limit value of the content of the pine bark extract in the composition for the oral cavity to be administered to the human oral cavity at one time may be determined in consideration of factors such as an amount of the extract that can be contained in the composition for the oral cavity and an amount of the extract that is not harmful to humans. More specifically, the composition for the oral cavity can be prepared such that the upper limit value of the content of the pine bark extract is, for example, 500 mg or less, preferably 300 mg or less, more preferably 150 mg or less, even more preferably 35 mg or less, and most preferably 20 mg or less. With this form, excellent effects of improving blood flow in the mouth and of reducing halitosis, for example, can be exerted, as shown in Examples.

It is preferable that the composition for the oral cavity of the present invention is prepared such that at least 2 mg of the pine bark extract is contained in an amount suitable for administration to the human oral cavity at one time. The “amount suitable for administration to the human oral cavity at one time” herein can be, in other words, an amount of the composition for the oral cavity that is suitable for putting in the human oral cavity, and preferably is an amount of the composition for the oral cavity that can be put in the human oral cavity without uncomfortable feeling. When the composition for the oral cavity is a liquid, the “amount suitable for administration to the human oral cavity at one time” refers to an amount of the composition for the oral cavity that is suitable for rinsing (washing) the oral cavity, for example. When the composition for the oral cavity is a solid, the “amount suitable for administration to the human oral cavity at one time” refers to an amount of the composition that can be ingested (chewed or licked) without uncomfortable feeling, for example. Thus, the “amount suitable for administration to the human oral cavity at one time” means, for example, one to several pieces in the case of ordinarily-sized tablets or troches.

Next, a preferable form in a case where the composition for the oral cavity is a solid will be described. When the composition for the oral cavity is a solid, a solid that is dissolved in the human oral cavity is preferable. Moreover, it is preferable that the composition for the oral cavity is prepared such that at least 2 mg, more preferably at least 3 mg, and even more preferably at least 4 mg of the pine bark extract is contained in the solid (the composition for the oral cavity), wherein the composition is suitable for administration to the human oral cavity at one time.

In a case where the composition for the oral cavity is a solid (e.g., tablets), it should be noted that when the solid is swallowed with water or the like and there is no or almost no retention time of the composition in the oral cavity, it is difficult to obtain an effect in the oral cavity. Thus, this form is not preferable for the composition for the oral cavity of the present invention. More specifically, a form in which the composition for the oral cavity (in particular, an active component thereof) can be applied (retained) to cells in the mouth for a predetermined time is preferable.

Next, a preferable form in a case where the composition for the oral cavity is a liquid will be described. When the composition for the oral cavity is a liquid, it is preferable that the composition contains a solvent. Examples of the solvent herein include water, ethanol, and aqueous ethanol. Moreover, when the composition for the oral cavity is a liquid, it is preferable that the concentration of the pine bark extract contained in the liquid is at least 0.2 (g/L). It would be appreciated that the concentration of the pine bark extract contained in the liquid may be at least 0.3 (g/L), or further may be at least 0.4 (g/L). With this form, more excellent effects of improving blood flow in the mouth and of reducing halitosis can be exerted, as shown in Examples. It should be noted that the “concentration of the pine bark extract contained in the liquid” herein can be expressed by formula (1) below.

$$Concentration ( g \text{/} L ) = \frac{\begin{matrix}Weight ( g ) of pine bark extract \\ contained in composition for oral cavity\end{matrix}}{Volume ( L ) of composition for oral cavity} \qquad formula ( 1 )$$

### EXAMPLES

Hereinafter, examples of the present invention will be described. However, the present invention is not limited to the examples below.

### Example 1

### Blood Flow Improvement Using Ethanol Aqueous Solution and Pine Bark Extract

First, 20 mg of a pine bark extract (ethanol extract of the bark of a pine, containing at least 20 wt % of OPCs, produced by TOYO SHINYAKU Co., Ltd.) was dissolved in a predetermined amount of a solvent (15% ethanol aqueous solution). Next, the solvent was added to the mixture so that the final volume was 100 mL, and thus a solution was prepared. Namely, a pine bark extract solution with a concentration of 0.2 (g/L) was prepared using 15% ethanol aqueous solution as the solvent. This solution was referred to as an Example 1A solution. It should be noted that the pine bark extract used herein is an extract that contains 50 wt % of proanthocyanidins, 30 wt % of OPCs, and 5 wt % of catechins with respect to the dry weight of the pine bark extract. Furthermore, the concentration (%) of the solvent (15% ethanol aqueous solution) used herein was based on volume/volume.

Separately, a pine bark extract solution with a concentration of 0.4 (g/L) was prepared using the same method as described above, except that the amount of the pine bark extract was 40 mg. This solution was referred to as an Example 1B solution. As a comparative example, a liquid containing only 15% ethanol aqueous solution (that is, the concentration of the pine bark extract was 0 (g/L)) was prepared. This solution was referred to as a Comparative Example 1 solution.

Next, using the Example 1A solution, the Example 1B solution, and the Comparative Example 1 solution (10 mL), an experiment was conducted with respect to the effect of improving blood flow in a tissue (the gingiva) of the mouth through the cooperation of four volunteers. More specifically, first, the volunteers put 10 mL of the solution in their mouths, moved the solution around in their mouths as appropriate so that the solution was spread over the entire oral cavity, and then ejected the solution. The time during which the solution was kept in the mouth was one minute. Next, at predetermined times (five minutes and 15 minutes) after the solution was ejected, the blood flow in the lower gingiva was measured with a blood flow meter. As the blood flow meter, a laser blood perfusion imager (PIM II; Perimed AB, Sweden) was used.

Table 1 shows the results of the blood flow measurement. The results of the blood flow measurement are shown as relative values, taking the measurement results of the blood flow measurement performed before putting the solutions in the mouth as 100. More specifically, larger values shown in Table 1 indicate a better blood flow.

TABLE 1

| | 5 min. | 15 min. |
|---|---|---|
| Ex. 1A solution | 132.62 ± 42.84 | 94.30 ± 31.80 |
| (concentration: 0.2 g/L) | | |
| Ex. 1B solution | 156.16 ± 41.41 | 112.81 ± 40.52 |
| (concentration: 0.4 g/L) | | |
| Com. Ex. 1 solution | 97.72 ± 31.76 | 90.61 ± 24.45 |
| (concentration: 0 g/L) | | |

Values in table are shown as mean ± standard deviation.

As shown in Table 1, it is recognized that at five minutes after the solutions were ejected, the result (mean: 132.62) obtained when putting the Example 1A solution in the mouth and the result (mean: 156.16) obtained when putting the Example 1B solution in the mouth were larger than the result (mean: 97.72) obtained when putting the Comparative Example 1 solution in the mouth. Thus, it is confirmed that the Example 1A solution and the Example 1B solution have the effect of improving (raising) blood flow in the mouth.

In the Example 1A solution, the concentration of the pine bark extract is 0.2 (g/L). In the Example 1B solution, the concentration of the pine bark extract is 0.4 (g/L). Thus, it is confirmed that in a case where the concentration of the pine bark extract is at least 0.2 (g/L), a certain level of effectiveness in improving blood flow in the mouth is obtained.

Focusing on the concentration of the pine bark extract, it is confirmed that the effect was obtained with a concentration of at least 0.2 (g/L), as described above. However, focusing on the amount of the pine bark extract, the following points can be confirmed. When the Example 1A solution (10 mL) was put in the mouth, 2 mg of the pine bark extract was contained in that solution that was put in the mouth. Thus, when putting in the mouth as in this example is also regarded as administration to a person, it can be also considered that in a case where 2 mg or more of the pine bark extract was administered, a certain level of effectiveness was obtained.

### Example 2

### Blood Flow Improvement Using Distilled Water and Pine Bark Extract

A solution was prepared using the same method as in the preparation of the Example 1A solution, except that distilled water was used as the solvent and 30 mg of the pine bark extract employed in Example 1 was used. Namely, an aqueous solution of the pine bark extract with a concentration of 0.3 (g/L) was prepared. This solution was referred to as an Example 2A solution.

Furthermore, a solution was prepared using the same method as the preparation method of the Example 2A solution, except that 40 mg of the pine bark extract was used. Namely, an aqueous solution of the pine bark extract with a concentration of 0.4 (g/L) was prepared. This solution was referred to as an Example 2B solution. As a comparative example, a liquid made of only distilled water was prepared. This liquid was referred to as a Comparative Example 2 liquid.

Next, using 10 mL of each of the Example 2A solution, the Example 2B solution, and the Comparative Example 2 liquid, an experiment was conducted with respect to the effect of improving blood flow in a tissue (the gingiva) of the mouth. It should be noted that this experiment was conducted using the same method as the method of Example 1, except that the number of volunteers was five.

Table 2 shows the results of the blood flow measurement. As in Example 1, the results of the blood flow measurement are shown as relative values, taking the measurement results of the blood flow measurement performed before putting the solutions in the mouth as 100. More specifically, larger values shown in Table 2 indicate a better blood flow.

TABLE 2

| | 5 min. | 15 min. |
|---|---|---|
| Ex. 2A solution | 123.15 ± 42.35 | 126.10 ± 54.65 |
| (concentration: 0.2 g/L) | | |
| Ex. 2B solution | 138.77 ± 42.99* | 123.15 ± 29.15** |
| (concentration: 0.4 g/L) | | |
| Com. Ex. 2 liquid | 89.11 ± 22.99 | 75.14 ± 21.39 |
| (concentration: 0 g/L) | | |

Values in table are shown as mean ± standard deviation.
*(p < 0.05)
**(p < 0.01)

As shown in Table 2, it is found that at five minutes after the solutions were ejected, the result (mean: 123.15) obtained when putting the Example 2A solution in the mouth and the result (mean: 138.77) obtained when putting the Example 2B solution in the mouth were larger than the result (mean: 89.11) obtained when putting the Comparative Example 2 liquid in the mouth. Thus, it is confirmed that the Example 2A solution and the Example 2B solution provide the effect of improving (raising) blood flow in the mouth. Furthermore, it is found that when distilled water was used as the solvent, even at 15 minutes after the solutions were ejected, the result (mean: 126.10) obtained when putting the Example 2A solution in the mouth and the result (mean: 123.15) obtained when putting the Example 2B solution in the mouth were larger than the result (mean: 75.14) obtained when putting the Comparative Example 2 liquid in the mouth. Thus, it is confirmed that the Example 2A solution and the Example 2B solution provide not only the effect of improving (raising) blood flow in the mouth but also the effect of sustaining this effect.

In the Example 2A solution, the concentration of the pine bark extract is 0.3 (g/L). In the Example 2B solution, the concentration of the pine bark extract is 0.4 (g/L). Thus, when distilled water is used as the solvent, it is confirmed that in a case where the concentration of the pine bark extract is at least 0.3 (g/L), a certain level of effectiveness in improving blood flow in the mouth is obtained. Furthermore, when putting in the mouth such as in this example is also regarded as administration to a person, it can be also considered as follows. When distilled water was used as the solvent, and in a case where at least 3 mg of the pine bark extract was administered, a certain level of effectiveness was obtained.

Furthermore, the results of this example were subjected to two-way analysis of variance (with repeated measures) on concentration and time. As a result, there were significant differences (p<0.01) with respect to the factor of concentration. There were significant differences (p<0.01) between the resultant value of the Example 2B solution (concentration: 0.4 g/L) and that of the Comparative Example 2 liquid (concentration: 0 g/L), and significant differences (p<0.05) between the resultant value of the Example 2A solution (concentration: 0.4 g/L) and that of the Comparative Example 2 liquid (concentration: 0 g/L).

Moreover, in the results of a paired t-test, at five minutes after the solutions were ejected, there were significant differences (p<0.05) between the resultant value of the Example 2B solution and that of the Comparative Example 2 liquid. Furthermore, at 15 minutes after the solutions were ejected, there were significant differences (p<0.01) between the resultant value of the Example 2B solution and that of the Comparative Example 2 liquid.

Herein, when the results of the Example 1B solution (15% alcohol solution, concentration: 0.4 g/L) and the results of the Comparative Example 2 liquid (aqueous solution, concentration: 0 g/L) were compared at five minutes after the solutions were ejected, there were significant differences (p<0.05) (Turkey-Kramer test).

### Example 3

### Blood Flow Improvement Using Tablets Containing Pine Bark Extract

Tableted products (tablets) were produced as the composition for the oral cavity containing the pine bark extract. Hereinafter, the method for producing the tablets will be described.

First, powders were obtained by using substances listed below and sufficiently blending the substances so that they were uniformly mixed. As the powders, three types of powders, i.e., a powder containing a pine bark extract, a powder containing a tea extract, and a powder containing no extract, were prepared.

<Formulation of Powder Containing Pine Bark Extract>

Granular sugar (FROST (registered trademark) sugar: produced by Nissin Sugar Manufacturing Co., Ltd.): 57 g

Lactose: 20 g

Maltitol: 20 g

Sucrose fatty acid ester: 2 g

Silicon dioxide: 1 g

Pine bark extract (the same extract as in Example 1): 0.4 g

<Formulation of Powder Containing Tea Extract>

Granular sugar: 57 g

Lactose: 20 g

Maltitol: 20 g

Sucrose fatty acid ester: 2 g

Silicon dioxide: 1 g

Tea extract (containing at least 90% of epigallocatechin gallate, product name “TEAVIGO” (registered trademark), produced by Roche Vitamin Japan): 0.4 g

<Formulation of Powder Containing no Extract>

Granular sugar: 57 g

Lactose: 20 g

Maltitol: 20 g

Sucrose fatty acid ester: 2 g

Silicon dioxide: 1 g

Next, tablets each weighing 250 mg were produced by tableting the powders obtained by the above-described formulations. More specifically, tablets containing the pine bark extract were produced from the powder containing the pine bark extract, tablets containing the tea extract were produced from the powder containing the tea extract, and tablets containing no extract were produced from the powder containing no extract, respectively. It should be noted that 0.996 mg (about 1 mg) of the extract was contained per tablet of containing the extract.

Next, using the tablets, an experiment was conducted with respect to the effect of improving blood flow in a tissue (the gingiva) of the mouth through the cooperation of four of the five volunteers in Example 1. More specifically, first, the volunteers put two tablets in their mouths, and ingested the tablets into the body while gradually dissolving the tablets in their mouths. Next, at predetermined times (five minutes and 15 minutes) after completion of ingestion of the tablets, the blood flow in the lower gingiva was measured with a blood flow meter. It should be noted that the blood flow meter and others that were used herein are the same as those in Example 1. Moreover, as an example of a substance that improves blood flow, vitamin E (100 mg) alone was ingested while being gradually dissolved in the mouth, and the blood flow was also measured. Furthermore, the tablets containing the pine bark extract (two tablets) were ingested by being swallowed at one gulp with 10 mL of distilled water, instead of being gradually dissolved in the mouth, and the blood flow was also measured.

Table 3 shows the results of the blood flow measurement. The results of the blood flow measurement are shown as relative values, taking the measurement results of the blood flow measurement performed before putting the tablets in the mouth as 100. In Table 3, the results when the tablets containing the pine bark extract were gradually dissolved in the mouth are shown in the field “pine bark extract”, the results for the tablets containing the tea extract are shown in the field “tea extract”, the results for the tablets containing no extract are shown in the field “no extract”, the results when vitamin E (100 mg) alone was ingested are shown in the field “vitamin E”, and the results when the tablets containing the pine bark extract (two tablets) were swallowed with distilled water are shown in the field “pine bark extract (swallowed)”.

Table 3 shows the results of the blood flow measurement. The results of the blood flow measurement are shown as relative values, taking the measurement results of the blood flow measurement performed before putting the tablets in the mouth as 100. In other words, larger values shown in Table 3 indicate a better blood flow. In Table 3, the results for the tablets containing the pine bark extract are shown in the field “pine bark extract”, the results for the tablets containing the tea extract are shown in the field “tea extract”, the results for the tablets containing no extract are shown in the field “no extract”, and the results when vitamin E (100 mg) alone was ingested are shown in the field “vitamin E”.

TABLE 3

| | 5 min. | 15 min. |
|---|---|---|
| Pine bark extract | 115.35 ± 6.87 | 111.55 ± 14.74 |
| Tea extract | 81.24 ± 7.75 | 98.95 ± 5.64 |
| Vitamin E | 116.99 ± 14.39 | 105.63 ± 16.16 |
| No extract | 96.84 ± 6.40 | 96.88 ± 13.38 |
| Pine bark extract | 97.74 ± 8.50 | 99.55 ± 10.80 |
| (swallowed) | | |

Values in table are shown as mean ± standard deviation.

As shown in Table 3, it is found that at five minutes after ingestion, the result (mean: 115.35) of “pine bark extract” is larger than the result (mean: 96.84) of “no extract” and the result (mean: 81.24) of “tea extract”. Thus, it is confirmed that the tablets containing the pine bark extract provide the effect of improving (raising) blood flow in the mouth. Furthermore, as for the result (mean: 116.99) of “vitamin E”, the result of “pine bark extract” was substantially equal to that of “vitamin E” (100 mg).

Furthermore, it is found that at 15 minutes after ingestion, the result (mean: 111.55) of “pine bark extract” is slightly larger than the result (mean: 96.88) of “no extract” and the result (mean: 105.63) of “vitamin E”. Thus, the tablets containing the pine bark extract provide the effect of sustaining the blood flow improving effect.

In the test of the tableted products, two tablets were ingested. This means that 2 mg of the pine bark extract was ingested. Thus, it can be considered that a certain level of effectiveness was obtained by ingesting 2 mg of the pine bark extract.

As shown in Table 3, when the tablets containing the pine bark extract (two tablets) were swallowed with distilled water, the result was close to the result of “no extract”, and substantially no effect was obtained. Furthermore, in the example in which the tablets containing the pine bark extract (two tablets) were swallowed with distilled water, the effect was not observed even at one hour after the tablets were ingested by swallowing. Thus, it is shown that when the composition for the oral cavity (solid containing 2 mg of pine bark extract) is swallowed with water or the like, it is difficult to obtain an effect in the oral cavity. In other words, in the case of the composition for the oral cavity containing pine bark extract in an amount as small as 2 mg, a form in which the composition can be applied (retained) to cells in the mouth for a predetermined time is preferable.

It should be noted that in the results of a mean difference test using the randomized blocks method on the individual volunteers and the ingested tablets and the like, there were significant differences (p<0.01) between the ingested tablets.

Furthermore, with respect to the palatability in a case where the tablets were dissolved in the mouth, all of the subjects replied that the tablets could be easily ingested because the astringent taste of the tablets was smaller than that of the solutions obtained in Example 2. Accordingly, it was found that the astringent taste of the pine bark extract is reduced when being ingested together with a sugar alcohol, and thus a composition suitable for administration in the oral cavity is obtained.

### Example 4

### Halitosis Reducing (Improving) Effect

A test was conducted with respect to the effect of reducing halitosis (garlic odor) through the cooperation of six to seven volunteers. The test method is described.

First, garlic paste was produced by putting one head of garlic and 50 mL of warmed distilled water in a mixer and grinding them. Next, a garlic mixed liquid was produced by mixing the obtained garlic paste (10 g) and warmed distilled water (5 g).

Next, the volunteers put the obtained garlic mixed liquid in their mouths, moved the mixed liquid around in their mouths as appropriate so that the mixed liquid was spread over the entire oral cavity, and then ejected the mixed liquid. The time during which the mixed liquid was kept in the mouth was 30 seconds.

Immediately after the mixed liquid was ejected, the volunteers put the tablets in their mouths and ingested the tablets while chewing them. At predetermined times after ejection of the mixed liquid, the amount (ppm) of methyl mercaptan contained in human breath was measured by blowing a predetermined amount of breath into a methyl mercaptan detector tube. It should be noted that “at predetermined times after” herein refers to after one minute, after five minutes, and after 10 minutes.

The tablets used in this test were tablets containing the pine bark extract (two tablets), tablets containing the tea extract (two tablets), and tablets containing no extract (two tablets), and the method for producing these tableted products was the same as the method described in Example 3. Furthermore, also a commercially available halitosis preventing agent (produced by Kobayashi Pharmaceutical Co., Ltd. “chewing breath care (registered trademark)”: one tablet) was tested.

Table 4 shows the results for the tablets containing the pine bark extract, Table 5 shows the results of the commercially available halitosis preventing agent, and Table 6 shows the results for the tablets containing the tea extract. The values shown in Tables 4 to 6 indicate the amount of methyl mercaptan contained in human breath that was blown into the detector tube. Thus, the smaller the values in the tables are (the greater the decreasing tendency is), the higher the effect of reducing halitosis is.

Furthermore, the values shown as “control” in each of the tables are the results for the tablets containing no extract. The results of “control” are shown in each of the tables in this manner because variations may be seen in the test results depending on the individual garlics used in the test. In other words, the results shown in the same table were obtained using the same garlic paste.

Table 7 shows mean and standard deviation of the values obtained from volunteers who ingested a product (i.e., tablets containing the pine bark extract, tablets containing the tea extract, or commercially available product) that possibly has a halitosis reducing effect, wherein each of the values was obtained by dividing the measured amount of methyl mercaptan with those of the control. In other words, a value shown in Table 7 smaller than one indicates that the halitosis reducing effect was obtained, and smaller values shown in Table 7 indicate a higher halitosis reducing effect.

TABLE 4

| | 1 min. | 5 min. | 10 min. |
|---|---|---|---|
| Pine bark extract | 8.90 ± 1.85 | 4.84 ± 3.05 | 0.94 ± 0.60 |
| Control | 9.49 ± 1.36 | 7.03 ± 2.43 | 4.52 ± 1.15 |

Values in table are shown as mean ± standard deviation.

TABLE 5

| | 1 min. | 5 min. | 10 min. |
|---|---|---|---|
| Commercially | 8.47 ± 1.83 | 7.63 ± 1.83 | 4.43 ± 2.68 |
| available product | | | |
| Control | 8.77 ± 1.11 | 7.00 ± 3.03 | 4.33 ± 1.48 |

Values in table are shown as mean ± standard deviation.

TABLE 6

| | 1 min. | 5 min. | 10 min. |
|---|---|---|---|
| Tea extract | 10.00 ± 0.00 | 8.90 ± 1.71 | 5.60 ± 1.95 |
| Control | 9.23 ± 1.20 | 7.07 ± 2.47 | 5.63 ± 3.62 |

Values in table are shown as mean ± standard deviation.
Value of tea extract (after 1 min.) shows that all the detection results were at or beyond the limit (10 ppm) of the measurement capability of the detector tube.

TABLE 7

| | 1 min. | 5 min. | 10 min. |
|---|---|---|---|
| Pine bark extract | 0.94 ± 0.15 | 0.64 ± 0.25 | 0.22 ± 0.16 |
| Tea extract | 1.10 ± 0.16 | 1.34 ± 0.37 | 1.35 ± 0.70 |
| Commercially | 0.98 ± 0.27 | 1.44 ± 1.17 | 1.11 ± 0.79 |
| available product | | | |

Values in table are shown as mean ± standard deviation.

As shown in Table 4, in comparison with the values of the control, the values of the pine bark extract are always smaller than the values of the control. Namely, it is found that the amount of methyl mercaptan detected in human breath was smaller in a case where the pine bark extract was ingested than in a case where the tablets containing no extract were ingested. Thus, it can be considered that a certain level of effectiveness in reducing halitosis was obtained by ingesting two tablets containing the pine bark extract, that is, 2 mg of the pine bark extract.

On the other hand, as shown in Tables 5 to 6, it is found that the values of the tea extract and the commercially available product are not smaller than the values of the control. Thus, it is shown that the tea extract and the commercially available product could not reduce the amount of methyl mercaptan detected in human breath in this test system.

Furthermore, as shown in Table 7, when the measured values of the amount of methyl mercaptan obtained in a case where the tablets containing the pine bark extract were ingested are divided by the measured values of the control, the resultant values (means) are significantly smaller than one. In other words, the effect of reducing halitosis was observed. On the other hand, in the case of the tea extract and the commercially available product, the means are larger than one or equivalent to one. Thus, the effect of reducing halitosis was not observed with the tea extract and the commercially available product in this test.

### INDUSTRIAL APPLICABILITY

As described above, the composition for the oral cavity of the present invention contains a pine bark extract that is extracted from the bark of a pine, and the pine bark extract contains at least 20 wt % of oligomeric proanthocyanidins with respect to the dry weight of the pine bark extract. This composition for the oral cavity is useful for preventing periodontal diseases by improving blood flow in the mouth. Furthermore, this composition for the oral cavity has the effect of reducing halitosis, and thus it is useful for preventing a person from making an unpleasant impression on others because of halitosis.

1. A composition for an oral cavity, comprising a pine bark extract that is extracted from a bark of a pine.

2. The composition for an oral cavity of claim 1, wherein the composition for an oral cavity is prepared such that at least 2 mg of the pine bark extract is administered to an oral cavity at one time.

3. The composition for an oral cavity of claim 1, further comprising a solvent, wherein the concentration of the pine bark extract is at least 0.2 (g/L).
